(19) United States
(12) Patent Application Publication
(10) Pub. No.: US 2020/0095643 A1
(43) Pub. Date: Mar. 26, 2020

(54) TIM-3 FOR ASSESSING THE SEVERITY OF CANCER

(71) Applicants: INSERM (INSTITUT NATIONAL DE LA SANTE ET DE LA RECHERCHE MEDICALE), Paris (FR); SORBONNE UNIVERSITE, Paris (FR); UNIVERSITE PARIS DIDEROT - PARIS 7, Paris (FR); UNIVERSITE PARIS DESCARTES, Paris (FR); ASSISTANCE PUBLIQUE HOPITAUX DE PARIS, Paris (FR)

(72) Inventors: Jérôme GALON, Paris (FR); Bernhard MLECNIK, Paris (FR); Gabriela BINDEA, Paris (FR)

(21) Appl. No.: 16/483,962

(22) PCT Filed: Feb. 7, 2018
(86) PCT No.: PCT/EP2018/053086

§ 371 Date: Aug. 6, 2019

(30) Foreign Application Priority Data
Feb. 7, 2017 (EP) 17305138.4

(51) Int. Cl. C12Q 1/6886

(52) U.S. Cl. CPC C12Q 1/6886; C12Q 2600/158; C12Q 2600/118

(57) ABSTRACT

The present invention relates to methods for assessing the severity of cancer by measuring the expression level of TIM-3 in a tumour sample. The present inventors have determined that the expression of TIM-3 (T-cell immunoglobulin and mucin-domain containing-3) can be correlated with the prognosis and with the responsiveness to treatment of patients suffering from solid cancer. They have particularly demonstrated that the specific ratio of the expression level of TIM-3 to the expression level of a biomarker of the adaptive immune response within the tumour is extremely predictive of patients' survival. Thus, the present invention relates to methods for determining the prognosis patients suffering from a solid cancer by determining the ratio of the expression level of TIM-3 to the expression level of a biomarker of the adaptive immune response in a tumour tissue sample obtained from said patient. Typically, these expression levels are determined by immunohistochemistry.

### FIELD OF THE INVENTION

The present invention relates to methods for assessing the severity of cancer by measuring the expression level of TIM-3 in a tumour sample.

### BACKGROUND OF THE INVENTION

Despite many advances in the development of treatments and of tests allowing a more accurate screening of patients, cancer is still a leading cause of death worldwide.

During the last few years, several studies have demonstrated that the intra-tumour immune microenvironment is strongly correlated with patients' survival (Galon et al., Immunity, vol. 39, 2013, Issue 1, pages 11-26). The identification of the immune contexture as an efficient predictive factor of cancer outcome led to the development of novel stratification systems allowing a more accurate prognosis of patients suffering from cancer (see e.g. EP patent No EP1943520 and EP patent applications No 13745117.5, 12 700 803.5 or 13701044.3).

Despite these advances, the Union for International Cancer Control still cites the UICC-TNM system as the gold standard method for staging cancer. The TNM (for “Tumour-Node-Metastasis”) staging system uses the size of the tumour, the presence or absence of tumour in regional lymph nodes, and the presence or absence of distant metastases, to assign a stage and an outcome to the tumour.

The TNM system has been developed from the observation that patients with small tumours have better prognosis than those with tumours of greater size at the primary site. In general, patients with tumours confined to the primary site have better prognosis than those with regional lymph node involvement, which in turn is better than for those with distant spread of disease from one body part two another. Accordingly, cancers are usually staged into four levels. Stage I cancer is a localized cancer with no cancer in the lymph nodes. Stage II cancer has spread near to where the cancer started. Stage III cancer has spread to lymph nodes. Stage IV cancer has spread to a distant part of the body. The assigned stage is used as a basis for selection of appropriate therapy and for prognostic purposes. Although useful, this staging method is imperfect and does not allow a reliable prognosis of cancer or an accurate prediction of the likeliness of a patient to respond to a particular treatment.

Accordingly, there is a continued need for more reliable biomarkers allowing an accurate evaluation of the prognosis of cancer and of the likeliness of said cancer to respond to known treatment.

### DETAILED DESCRIPTION

The present inventors have determined that the expression of TIM-3 (T-cell immunoglobulin and mucin-domain containing-3) can be correlated with the prognosis of patients suffering from solid cancer. They have particularly demonstrated that the specific ratio of the expression level of TIM-3 to the expression level of a biomarker of the adaptive immune response within the tumour is extremely predictive of patients' survival.
Prognosing cancer by measuring the intra-tumour TIM-3/(biomarker of the adaptive immune response) ratio

Thus, in a first aspect the present invention relates to a method for determining the prognosis of a patient suffering from a solid cancer comprising a step of determining, in a tumour tissue sample obtained from said patient, the ratio of the expression level of TIM-3 to the expression level of a biomarker of the adaptive immune response.

The present inventors have particularly determined that:
a high ratio of the expression of TIM-3 to the expression of a biomarker of the adaptive immune response in the centre of the tumour (CT) is associated with a good prognosis; whereas
a high ratio of the expression of TIM-3 to the expression of a biomarker of the adaptive immune response in the invasive margin of the tumour (IM) is associated with a poor prognosis.

Thus, the present invention particularly relates to a method for determining the prognosis of a patient suffering from a solid cancer comprising the steps of:
i) determining the expression level of TIM-3 and that of a biomarker of the adaptive immune response in the centre of the tumour (CT) in a tumour tissue sample obtained from said patient;
ii) determining the ratio of the expression level of TIM-3 to the expression level of said biomarker in the centre of the tumour;
iii) comparing said ratio with a predetermined reference value;
iv) providing a good prognosis when said ratio is higher than the predetermined reference value.

The present invention further relates to a method for determining the prognosis of a patient suffering from a solid cancer comprising the steps of:
i) determining the expression level of TIM-3 and that of a biomarker of the adaptive immune response in the invasive margin (IM) in a tumour tissue sample obtained from said patient;
ii) determining the ratio of the expression level of TIM-3 to the expression level of said biomarker in the invasive margin of the tumour;
iii) comparing said ratio with a predetermined reference value;
iv) providing a bad prognosis when said ratio is higher than the predetermined reference value.

In a further aspect, the present invention also relates to a method for determining the prognosis of a patient suffering from a solid cancer comprising the steps of:
i) determining the expression level of TIM-3 and the expression level of a biomarker of the adaptive immune response in the centre of the tumour (CT) and in the invasive margin of the tumour (IM) in a tumour tissue sample obtained from said patient;
ii) determining the ratio of the expression level of TIM-3 to the expression level of said biomarker in the centre of the tumour (CT ratio) and in the invasive margin of the tumour (IM ratio);
iii) comparing said CT ratio and IM ratio with reference values determined for the centre of the tumour (CT reference value) and for the invasive margin of the tumour (IM reference value), respectively;
iv) providing:
   a good prognosis when the CT ratio is higher than the CT reference value, and the IM ratio is lower than the IM reference value;
   an intermediate prognosis when the CT ratio and the IM ratio are both higher than the CT reference value and IM reference value, respectively, or when the CT ratio and the IM ratio are both lower than the CT reference value and IM reference value, respectively; and
   a bad prognosis when the CT ratio is lower than the CT reference value, and the IM ratio is higher than the IM reference value.

TIM-3 (T-cell immunoglobulin and mucin-domain containing-3), also known as HAVCR2 (Hepatitis A virus cellular receptor 2), is a cell surface receptor implicated in modulating innate and adaptive immune responses (immune checkpoint). TIM-3 is encoded by the HAVCR2 gene in humans (which has the HGNC reference No 18437 in the Hugo Gene Nomenclature Committee Database). Generally identified as negatively regulating the immune responses, it is commonly associated with T-cells exhaustion during cancer.

A biomarker of the adaptive immune response refers to any biomarker that is expressed by a cell that is an actor of the adaptive immune response in the tumour or that contributes to the settlement of the adaptive immune response in the tumour. Such biomarkers are fully disclosed in the international patent publication No WO2007/045996. The adaptive immune response, also called “acquired immune response”, comprises antigen-dependent stimulation of T cell subtypes, B cell activation and antibody production. Cells of the adaptive immune response include but are not limited to cytotoxic T cells, memory T cells, Th1 and Th2 cells and B-cells. Furthermore, additional cells such as activated macrophages, activated dendritic cells, NK cells and NKT cells can also drive, favor or induce the adaptive response and are thus indirectly involved into the adaptive immunity. Accordingly, a biomarker representative of the adaptive immune response may be e.g. selected from the cluster of the co-modulated genes for the Th1 adaptive immunity, for the cytotoxic response, or for the memory response, and may be a T-cell surface marker, a Th1 cell surface marker, an interleukin (or an interleukin receptor), or a chemokine or (a chemokine receptor).

In a particular embodiment, the biomarker of the adaptive immune response is selected from CD3, CD8, CD45RO, CD20, CD103, CD19 and CD4.

CD3 (cluster of differentiation 3), is cell surface receptor expressed by T-lymphocytes. It associates with the T-cell receptor (TCR) so as to induce T-cell activation and proliferation.

CD8 (cluster of differentiation 8), is cell surface receptor expressed by cytotoxic T-lymphocytes. CD8 serves as a co-receptor for the T cell receptor (TCR).

CD45RO is the shortest isoform of CD45 (cluster of differentiation 45, also known as Protein tyrosine phosphatase, receptor type, C [PTPRC]). It is a transmembrane protein expressed by memory T cells and it facilitates T cell activation.

CD20 (cluster of differentiation 20) is a B-lymphocyte surface molecule (B-cell biomarker). CD20 is encoded by the MS4A1 gene in human (which corresponds to the Ensembl gene reference No ENSG00000156738). Its function is to enable the B-cell immune response, particularly against T-independent antigens.

CD103 (cluster of differentiation 103), also known as Integrin, alpha E (ITGAE) is an integrin protein that is encoded by the ITGAE gene in human (which corresponds to the Ensembl gene reference No ENSG00000083457). CD03 combines with the beta 7 integrin to form the E-cadherin binding integrin known as the human mucosal lymphocyte-1 antigen.

CD19 (cluster of differentiation 19) is a cell surface molecule expressed on B-cells and follicular dendritic cells. It is encoded by the CD19 gene (Ensembl Ref. No ENSG00000177455). CD19 assembles with the B-cell receptor so as to decrease the threshold for antigen receptor-dependent stimulation.

CD4 (cluster of differentiation 4) is a glycoprotein found on the surface of immune cells such as T helper cells, monocytes, macrophages, and dendritic cells. It is a co-receptor that assists the TCR in communicating with antigen-presenting cells.

In one embodiment, the expression level of one biomarker of the adaptive immune response is measured. However, in another embodiment, the expression level of 1, 2, 3, 3, 4, 5, 6 or 7 biomarker(s) of the adaptive immune response can be measured.

The expression level TIM-3 and of the biomarker of the adaptive immune response can be measured by several techniques which are routine to the skilled person.

In one embodiment, the expression level of TIM-3 and of the biomarker of the adaptive immune response is measured by determining the density of cells expressing these molecules, i.e., by measuring the density of TIM-3+ cells and that of cells expressing the said biomarker (such as e.g. CD3+ cells when the biomarker of the adaptive immune response is CD3).

Typically, methods for measuring the density of these cells comprise a step of contacting the tumour tissue sample with at least one selective binding agent capable of selectively interacting with TIM-3, or with the selected biomarker of the adaptive immune response. The selective binding agent may be a polyclonal antibody or a monoclonal antibody, an antibody fragment, synthetic antibodies, or other protein-specific agents such as nucleic acid or peptide aptamers.

The skilled person knows several antibodies which are specific to TIM-3, to CD3, CD8, or to any other biomarker according to the present invention. Many of these antibodies are commercially available. In order to be detected by microscopy or by an automated analysis system, the antibody may be directly tagged with detectable labels such as enzymes, chromogens or fluorescent probes or indirectly detected with a secondary antibody conjugated with detectable labels.

Immunohistochemistry is particularly suitable for carrying out the method according to the present invention. Typically, the tissue tumour sample is firstly incubated with labelled antibodies directed against TIM-3 and/or against the biomarker of the adaptive immune response according to the present invention. After washing, the labelled antibodies which are bound to these markers are revealed by the appropriate technique, depending of the kind of label born by the labelled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems.

The density TIM-3+ cells and of the cells expressing the biomarker according to the present invention may be expressed as the number of these cells that are counted per one unit of surface area of tissue sample, e.g. as the number of TIM-3+, CD3+, CD8+, CD45RO+ cells (etc.) that are counted per cm² or mm² of surface area of tumour tissue sample. It may also be expressed as the number of TIM-3+, CD3+, CD8+, CD45RO+ cells (etc.) per one volume unit of sample, e.g. as the number of these cells per cm3 of tumour tissue sample. The density of TIM-3+ cells and of e.g., CD3+, CD8+, CD45RO+ cells may also consist of the percentage of TIM-3+, CD3+, CD8+, CD45RO+ cells per total cells (set at 100%).

The density may be measured in the “cold spot”, i.e., in the regions of the tumor sample where the density is the lowest, or in the 2, 3, 4, 5, 6, 7, 8, 9, 10 “cold spots”, corresponding to the 2 to 10 area with the lowest densities.

The density may also be measured in the “hot spot”, i.e., in the regions where the density is the highest, or in the 2, 3, 4, 5, 6, 7, 8, 9, 10 “hot spots”, corresponding to the 2 to 10 area with the highest densities.

One can also determine the mean density on the whole tumor sample.

In another embodiment, the density of TIM-3+ cells and of the cells expressing the biomarker according to the present invention can also be determined by measuring the expression level of TIM-3 and of the biomarker of the adaptive immune response in the “target” part of the tumour sample (e.g. the centre of the tumour), and by normalizing the results in function of the number of cells present in the sample. The skilled person knows several techniques which allow determining the expression level of a gene (see below).

Thus, in another embodiment, the expression level of TIM-3 and of the biomarker according to the present invention is determined by measuring the expression level of the genes encoding these molecules.

Typically, an expression level of a gene is assessed by determining the quantity of mRNA produced by this gene. Methods for determining a quantity of mRNA are well known in the art. For example nucleic acid contained in the samples (e.g., cell or tissue prepared from the patient) is first extracted according to standard methods, for example using lytic enzymes or chemical solutions or extracted by nucleic-acid-binding resins following the manufacturer's instructions.

The thus extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA), quantitative new generation sequencing of RNA (NGS).

Nucleic acids (polynucleotides) comprising at least 10 nucleotides and exhibiting sequence complementarity or homology to the mRNA of interest herein find utility as hybridization probes or amplification primers. It is understood that such nucleic acids need not be completely identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes comprise single-stranded nucleic acids of between 10 to 1000, typically of between 20 and 500 nucleotides. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6× SCC. SCC is a 0.15 M NaCl, 0.015 M Na-citrate).

In a particular embodiment, the methods of the invention comprise the steps of providing total RNAs extracted from cells from the center or from the invasive margin of a tumor sample, and subjecting the RNAs to amplification and hybridization to specific probes, more particularly by means of a quantitative or semi-quantitative RT-PCR.

Probes made according the disclosed methods can be used for nucleic acid detection, such as in situ hybridization (ISH) procedures (for example, fluorescence in situ hybridization (FISH), chromogenic in situ hybridization (CISH) and silver in situ hybridization (SISH)) or comparative genomic hybridization (CGH). Numerous procedures for ISH, FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. Probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other embodiments, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH).

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

The expression level of a gene may be expressed as absolute expression level or normalized expression level. Both types of values may be used in the present method. The expression level of a gene is preferably expressed as normalized expression level when quantitative PCR is used as method of assessment of the expression level because small differences at the beginning of an experiment could provide huge differences after a number of cycles.

Typically, expression levels are normalized by correcting the absolute expression level of a gene by comparing its expression to the expression of a gene that is not relevant for determining the prognosis of the patient, e.g., a housekeeping gene that is constitutively expressed. Suitable genes for normalization include housekeeping genes such as the actin gene ACTB, ribosomal 18S gene, GUSB, PGK1 and TFRC. This normalization allows comparing the expression level of one sample, e.g., a patient sample, with the expression level of another sample, or comparing samples from different sources.

In a further embodiment, the expression level of the target genes can be measured by “digital gene expression assay” (Nanostring Technologies). This technique utilizes a digital color-coded barcode technology based on direct multiplexed measurement of gene expression. It uses molecular “barcodes” and single molecule imaging to detect and count transcripts in a single reaction (see e.g. Geiss et al., Nat Biotechnol. 2008 March; 26(3):317-25).

The claimed invention implies a step of comparing the ratio of the expression level of TIM-3+ to that of a biomarker of the adaptive immune response determined in the tumour sample with a predetermined reference value. The present application comprise examples disclosing how to determine the “reference value” and how to use it according to the present invention.

Such predetermined reference value may consist of a “cut-of” value that may be determined as described hereunder. A cut-off value may be predetermined by carrying out a method comprising the steps of:

a) providing a collection of tumour tissue samples from cancer patients;

b) providing, for each tumour tissue sample provided at step a), information relating to the actual prognosis for the corresponding cancer patient (i.e. the duration of the disease-free survival (DFS) or the overall survival (OS));

c) providing a serial of arbitrary quantification values;

d) determining the TIM-3/(biomarker of the adaptive immune response) ratio in the whole tumour or at the invasive margin of the tumour or in the centre of the tumour for each tumour tissue sample contained in the collection provided at step a);

e) classifying said tumour tissue samples in two groups for one specific arbitrary quantification value provided at step c), respectively: (i) a first group comprising tissue tumour samples that exhibit a quantification value for said ratio that is lower than the said arbitrary quantification value contained in the said serial of quantification values; (ii) a second group comprising tumour tissue samples that exhibit a quantification value said ratio that is higher than the said arbitrary quantification value contained in the said serial of quantification values; whereby two groups of tumour tissue samples are obtained for the said specific quantification value, wherein the tumours tissue samples of each group are separately enumerated;

f) calculating the statistical significance between (i) the quantification value obtained at step e) and (ii) the actual clinical outcome of the patients from which tumour tissue samples contained in the first and second groups defined at step f) derive;

g) reiterating steps f) and g) until every arbitrary quantification value provided at step d) is tested;

h) setting the said predetermined reference value (“cut-off” value) as consisting of the arbitrary quantification value for which the highest statistical significance (most significant) has been calculated at step g).

As it is disclosed above, said method allows the setting of a single “cut-off” value permitting discrimination between poor and good prognosis. Practically, as it is disclosed in the examples herein, high statistical significance values (e.g. low P values) are generally obtained for a range of successive arbitrary quantification values, and not only for a single arbitrary quantification value. Thus, in one alternative embodiment of the method of determining “cut-off” values above, a minimal statistical significance value (minimal threshold of significance, e.g. maximal threshold P value) is arbitrarily set and the range of arbitrary quantification values for which the statistical significance value calculated at step g) is higher (more significant, e.g. lower P value) are retained, whereby a range of quantification values is provided. Said range of quantification values consist of a “cut-off” value according to the invention. According to this specific embodiment of a “cut-off” value, poor or good prognosis can be determined by comparing the TIM-3/(biomarker of the adaptive immune response) ratio determined at step i) with the range of values delimiting the said “cut-off” value. In certain embodiments, a cut-off value consisting of a range of quantification values consists of a range of values centred on the quantification value for which the highest statistical significance value is found (e.g. generally the minimum P value which is found).

Typically, the predetermined reference value may consist of the TIM-3/(biomarker of the adaptive immune response) value that correlates with a poor prognosis (e.g. a short disease-free survival time), or in contrast may consist of the TIM-3/(biomarker of the adaptive immune response) value that correlates with good prognosis (e.g. a long disease-free survival time).

The “tumour sample” can be obtained from any tissue sample derived from the tumour of the patient. The tissue sample is obtained for the purpose of the in vitro evaluation. The sample can be fresh, frozen, fixed (e.g., formalin fixed), or embedded (e.g., paraffin embedded). In a particular embodiment the sample results from biopsy performed in a tumour sample of the patient. An example is an endoscopic biopsy performed in the bowel of the patient suffering from colorectal cancer.

The tumour tissue sample can be a “whole” tumour tissue sample, or can alternatively be either a tissue obtained from the centre of the tumour (CT) or from the tissue directly surrounding the tumour (i.e. the invasive margin [IM] of the tumour).

Typically, in the context of the present invention, the patient suffering from cancer is human.

Typically, the methods of the invention apply to various organs of cancer origin (such as breast, colon, rectum, lung, head and neck, bladder, ovary, prostate), and also to various cancer cell types (adenocarcinoma, squamous cell carcinoma, large cell cancer, melanoma, etc).

In a particular embodiment, the patient suffers from a solid cancer selected from the group consisting of adrenal cortical cancer, anal cancer, bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), sarcomas such as liposarcoma and soft-tissue sarcoma, brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyo sarcoma (e.g. embryonal rhabdomyo sarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

In a more particular embodiment, the cancer is a colorectal cancer.

The present invention also relates to a kit for performing the methods of the invention, wherein said kit comprises means for specifically measuring the expression level of TIM-3 and the expression level of a biomarker of the adaptive immune response according to the present invention. The “means” can be e.g. antibodies directed against TIM-3 and against said biomarker of the adaptive immune response, and are further labelled so as to allow detecting the cells expressing TIM-3 and/or said biomarker. In one embodiment, said antibodies are directed against CD3, CD8, CD45RO, CD4, CD20, CD19 or CD103 and are labelled so as to allow detecting TIM-3+, CD3+, CD8+, CD45RO+, CD4+, CD20+, CD19+ or CD103+ cells.
TIM-3 alone allows determining the outcome of solid cancer and predicting response to treatment

Surprisingly, the inventors have further observed that, contrary to the common belief, a high density of TIM-3 within the tumour is generally associated with a good prognosis.

Thus, in a further aspect, the present invention relates to a method for determining the prognosis of a patient suffering from solid cancer comprising the steps of:
i) measuring, in a tumour tissue sample obtained from said patient, the expression level of TIM-3;
ii) comparing said TIM-3 expression level with a predetermined reference value;
v) providing a good prognosis when expression level of TIM-3 is higher than the predetermined reference value.

In a particular embodiment, the expression level of TIM-3 is measured at the centre of the tumour.

Typically, the expression level and the reference level are determined as explained above.

In a further embodiment, the prognosis of said patient is determined by measuring the expression level of TIM-3 and by further measuring the expression level of a biomarker of the adaptive immune response as disclosed above. Such a biomarker is e.g. selected from CD3, CD8, CD45RO, CD4, CD19, CD20 and CD103.

Thus, according to a particular embodiment, the present invention further relates to a method for determining the prognosis of a patient suffering from solid cancer comprising the steps of:
i) measuring, in a tumour tissue sample obtained from said patient; the expression level of TIM-3 and that of a biomarker of the adaptive immune response;
ii) comparing said TIM-3 expression level and said biomarker expression level with predetermined reference values;
v) providing a good prognosis when both the expression level of TIM-3 and of the biomarker are higher that their predetermined reference values.

The inventors further demonstrated that the expression level of TIM-3 combined with that of a biomarker of the adaptive immune response allows determining whether a patient is susceptible to respond to anticancer treatment, i.e. that the patient would advantageously receive an anticancer treatment. They particularly demonstrated that patients presenting a high intra-tumour TIM-3 expression level and a high intra-tumour expression level of a biomarker of the adaptive immune response had a significantly increased overall survival after treatment than patients who do not present a high TIM-3 expression level and a high expression level of said biomarker within their tumour.

Accordingly, the present invention further provides a method for determining whether a patient suffering from solid cancer will advantageously receive an anticancer treatment, said method comprising the steps of:
i) measuring, in a tumour tissue sample obtained from said patient, the expression level of TIM-3 and that of a biomarker of the adaptive immune response;
ii) comparing said TIM-3 expression level and said biomarker expression level with predetermined reference values;
iii) assessing that the patient will advantageously receive an anticancer treatment when both the expression level of TIM-3 and of said biomarker are higher that their predetermined reference values.

The biomarker of the adaptive immune response is as disclosed above.

An anti-cancer treatment may consist of radiotherapy, chemotherapy or immunotherapy. The treatment may consist of an adjuvant therapy (i.e. treatment after chirurgical resection of the primary tumor) of a neoadjuvant therapy (i.e. treatment before chirurgical resection of the primary tumor). The above method allows predicting the response to all these types of treatments because it allows determining the immune contexture of the tumour and thus the susceptibility of a tumour to be treated, regardless of the type of treatment.

The present invention therefore relates to a chemotherapeutic agent, a radiotherapeutic agent, or an immunotherapeutic agent, preferably the latter, for use in the treatment of a patient suffering from solid cancer for whom it has been considered that he would advantageously receive anti-cancer treatment according to the above method of the invention.

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycopheno lie acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1 ; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the patient with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-a), IFN-beta (IFN-beta) and IFN-gamma (IFN-y). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). The use of IFN-alpha, alone or in combination with other immunotherapeutics or with chemotherapeutics, has shown efficacy in the treatment of various cancers including melanoma (including metastatic melanoma), renal cancer (including metastatic renal cancer), breast cancer, prostate cancer, and cervical cancer (including metastatic cervical cancer).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Interleukins, alone or in combination with other immunotherapeutics or with chemotherapeutics, have shown efficacy in the treatment of various cancers including renal cancer (including metastatic renal cancer), melanoma (including metastatic melanoma), ovarian cancer (including recurrent ovarian cancer), cervical cancer (including metastatic cervical cancer), breast cancer, colorectal cancer, lung cancer, brain cancer, and prostate cancer.

Interleukins have also shown good activity in combination with IFN-alpha in the treatment of various cancers (Negrier et al., Ann Oncol. 2002 13(9):1460-8 ; Touranietal, J. Clin. Oncol. 2003 21(21):398794).

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in patients undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Colony stimulating factors have shown efficacy in the treatment of cancer, including melanoma, colorectal cancer (including metastatic colorectal cancer), and lung cancer.

Non-cytokine adjuvants suitable for use in the combinations of the present invention include, but are not limited to, Levamisole, alum hydroxide (alum), Calmette-Guerin bacillus (ACG), incomplete Freund's Adjuvant (IFA), QS-21, DETOX, Keyhole limpet hemocyanin (KLH) and dinitrophenyl (DNP). Non-cytokine adjuvants in combination with other immuno- and/or chemotherapeutics have demonstrated efficacy against various cancers including, for example, colon cancer and colorectal cancer (Levimasole); melanoma (BCG and QS-21); renal cancer and bladder cancer (BCG).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Monoclonal antibodies are used in the treatment of a wide range of cancers including breast cancer (including advanced metastatic breast cancer), colorectal cancer (including advanced and/or metastatic colorectal cancer), ovarian cancer, lung cancer, prostate cancer, cervical cancer, melanoma and brain tumours. Other examples include anti-CTLA4 antibodies (e.g. Ipilimumab), anti-PD1 antibodies, anti-PDL1 antibodies, anti-TIMP3 antibodies, anti-LAG3 antibodies, anti-B7H3 antibodies, anti-B7H4 antibodies or anti-B7H6 antibodies, anti-TIM-3 antibodies.

In a particular embodiment, the immunotherapeutic agent is an anti-TIM-3 antibody.

Monoclonal antibodies can be used alone or in combination with other immunotherapeutic agents or chemotherapeutic agents.

Active specific immunotherapy typically involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

The immunotherapeutic treatment may consist of an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the patient's circulating lymphocytes, or tumor infiltrated lymphocytes, are isolated in vitro, activated by lymphokines such as IL-2 or transuded with genes for tumor necrosis, and readministered (Rosenberg et al., 1988; 1989). The activated lymphocytes are most preferably be the patient's own cells that were earlier isolated from a blood or tumor sample and activated (or “expanded”) in vitro. This form of immunotherapy has produced several cases of regression of melanoma and renal carcinoma.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

The invention will be further illustrated in the following examples.

### BRIEF DESCRIPTION OF THE FIGURES

FIG. 1: TIM-3+ cells were quantified in situ by immunohistochemistry. TIM-3 specific immunohistochemistry were performed, and positive cells (Brown DAB positive cells), were quantified and their density recorded (cells/mm2). Measurements were performed in the Centre (CT) of the tumour (A, C), and in the invasive margin (IM) of the tumour (B, D).

High TIM-3 expression quantified in CT or IM correlated with good clinical outcome (A, B). Similar results for PFS (progression-free survival) DFS (disease-free survival) and OS (overall survival). CD3 positive cells were quantified in the same manner by immunohistochemistry, and densities of CD3+ cells were recorded. A high ratio of TIM-3 to CD3 in the CT is associated with good survival (C). In contrast, a high ratio of TIM-3 to CD3 in the IM is associated with poor survival (D).

FIG. 2: Colon cancer patients stages I-IV (n=70) were categorized into 2 groups based on based on the CD8A and TIM-3 (HAVR2) expression level. Kaplan-Meier curves of disease-free survival are represented. Cytotoxic T-cells gene (CD8A) and TIM-3 gene (HAVCR2) expression were measured, and categorized in high expression (Hi) or low expression (Lo) using the optimal-P-value cutoff. Patients having both high expression level of CD8A and HAVCR2 were grouped (HiHi), and all other patients were grouped (Others). Patients with high expression level of CD8A and HAVCR2 (HiHi), had better prognosis (P<0.05).

FIG. 3: Colon cancer patients stages I-IV (n=145) were categorized into 2 groups based on based on the CD8A and TIM-3 (HAVR2) expression level. Kaplan-Meier curves of disease-free survival are represented. Cytotoxic T-cells gene (CD8A) and TIM-3 gene (HAVCR2) expression were measured, and categorized in high expression (Hi) or low expression (Lo) using the optimal-P-value cutoff. Patients having both high expression level of CD8A and HAVCR2 were grouped (HiHi), and all other patients were grouped (Others). Patients with high expression level of CD8A and HAVCR2 (HiHi), had better prognosis (P<0.05).

FIG. 4: Breast cancer patients (familial breast cancer) (n=183) were categorized into 2 groups based on based on the CD8A and TIM-3 (HAVR2) expression level. Kaplan-Meier curves of disease-free survival are represented. Cytotoxic T-cells gene (CD8A) and TIM-3 gene (HAVCR2) expression were measured, and categorized in high expression (Hi) or low expression (Lo) using the optimal-P-value cutoff. Patients having both high expression level of CD8A and HAVCR2 were grouped (HiHi), and all other patients were grouped (Others). Patients with high expression level of CD8A and HAVCR2 (HiHi), had better prognosis (P<0.05).

FIG. 5: Breast cancer patients (Early stage invasive) (n=241) were categorized into 2 groups based on based on the CD8A and TIM-3 (HAVR2) expression level. Kaplan-Meier curves of disease-free survival are represented. Cytotoxic T-cells gene (CD8A) and TIM-3 gene (HAVCR2) expression were measured, and categorized in high expression (Hi) or low expression (Lo) using the optimal-P-value cutoff. Patients having both high expression level of CD8A and HAVCR2 were grouped (HiHi), and all other patients were grouped (Others). Patients with high expression level of CD8A and HAVCR2 (HiHi), had better prognosis (P<0.05).

FIG. 6: Colon cancer patients (n=589) were categorized into 4 groups. Kaplan-Meier curves of disease-free survival are represented. Patients treated with adjuvant chemotherapy (YES) or not treated with adjuvant chemotherapy (NO), and patients categorized based on the CD8A and TIM-3 (HAVR2) expression level. Cytotoxic T-cells gene (CD8A) and TIM-3 gene (HAVCR2) expression were measured, and categorized in high expression (Hi) or low expression (Lo) using the optimal-P-value cutoff. Patients having both high expression level of CD8A and HAVCR2 were grouped (HiHi), and all other patients were grouped (Others). Patients with high expression level of CD8A and HAVCR2 (HiHi), had better response to adjuvant chemotherapy (P<0.05).

FIG. 7: Colon cancer patients (n=37) were categorized into 4 groups. Kaplan-Meier curves of overall survival are represented. Patients treated with adjuvant chemotherapy (YES) or not treated with adjuvant chemotherapy (NO), and patients categorized based on the CD8A and TIM-3 (HAVR2) expression level. Cytotoxic T-cells gene (CD8A) and TIM-3 gene (HAVCR2) expression were measured, and categorized in high expression (Hi) or low expression (Lo) using the optimal-P-value cutoff. Patients having both high expression level of CD8A and HAVCR2 were grouped (HiHi), and all other patients were grouped (Others). Patients with high expression level of CD8A and HAVCR2 (HiHi), had better response to adjuvant chemotherapy (P<0.05).

### EXAMPLES

### Example 1: The Ratio of the Expression Level of TIM-3 to the Expression Level of CD3, CD8, CD45RO or CD4 is Predictive of a Patient's Prognosis

Tissue microarray from the center (CT) and invasive margin (IM) of colorectal tumors (n=107) were constructed. Assessment of the invasive margin area was performed on standard paraffin sections and was based on the histomorphological variance of the tissue. The invasive margin was defined as a region centered on the border separating the host tissue from malignant glands, with the extend of 1 mm. TMA sections were incubated (60 min. at room temperature) with monoclonal antibodies against CD3 and TIM3. Envision+system (enzyme-conjugated polymer backbone coupled to secondary antibodies) (Dako, Glostrup, Denmark) and DAB-chromogen were applied (Dako, Glostrup, Denmark). Double stainings were revealed with phosphate-conjugated secondary antibodies and FastBlue-chromogen. For single stainings, tissue sections were counterstained with Harris hematoxylin (Sigma Aldrich Saint Louis, MO). Isotype-matched mouse monoclonal antibodies were used as negative controls. Slides were analyzed using an image analysis workstation (Spot Browser, Excilone, Elancourt, France). Polychromatic high-resolution spot-images (740×540 pixel, 1.181 μm/pixel resolution) were obtained (x200 fold magnification). The density was recorded as the number of positive cells per unit tissue surface area.

CD3 and TIM3 densities were quantified in the center (CT) and invasive margin (IM) of the tumor. The survival of patients with high (Hi) TIM3 (at optimal cut-off) was compared with the low (Lo) TIM3 patients (Kaplan-Meier (KM) curves for Disease-Free-Survival). In addition, The ration TIM3 vs CD3 was calculated for each patient. The survival of patients with high (Hi) TIM3/CD3 ratio (at optimal cut-off) was compared with the low (Lo) TIM3/CD3 ratio patients (Kaplan-Meier (KM) curves for Disease-Free-Survival). A logrank p-value smaller than 0.05 was considered significant.

We demonstrate that the ratio of the expression level of TIM-3 to expression level of a biomarker of the adaptive immune response in a tumour tissue sample is strongly correlated with the outcome of cancer.

In this Example, TIM-3+ cells were quantified in situ by immunohistochemistry. TIM-3 specific immunohistochemistry were performed, and positive cells (Brown DAB positive cells), were quantified and their density recorded (cells/mm2). Measurements were performed in the Centre (CT) of the tumour and in the invasive margin (IM) of the tumour. CD3 positive cells were quantified in the same manner by immunohistochemistry, and densities of CD3+ cells were recorded.

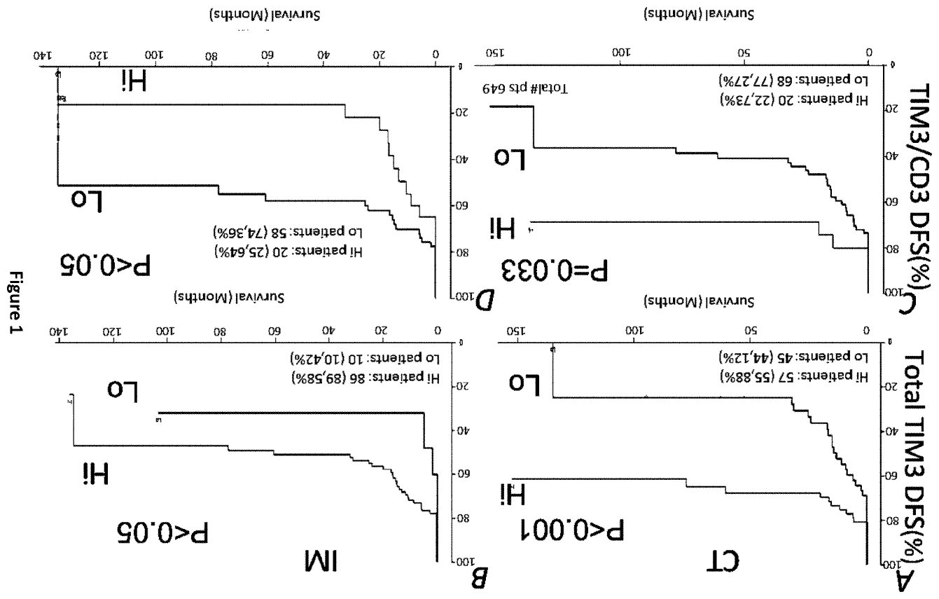

As shown in FIG. 1, high TIM-3 expression quantified in CT or IM correlated with good clinical outcome (A, B). Similar results for PFS (progression-free survival) DFS (disease-free survival) and OS (overall survival). A high ratio of TIM-3 to CD3 in the CT is associated with good survival (C). In contrast, a high ratio of TIM-3 to CD3 in the IM is associated with poor survival (D).

### Example 2: The Expression Level of TIM-3 alone or in Combination with the Expression Level of a Biomarker of the Adaptive Immune Response is Predictive of a Patient's Prognosis

1) Material, methods for gene expression (Affymetrix Human Genome U133 Plus 2.0 Array). The tissue sample material (n=105) was snap-frozen within 15 minutes after surgery and stored in liquid nitrogen. Frozen tumour specimens were randomly selected for RNA extraction. The total RNA was isolated by homogenization with the RNeasy isolation kit (Qiagen, Valencia, Calif.). A bioanalyzer (Agilent Technologies, Palo Alto, Calif.) was used to evaluate the integrity and the quantity of the RNA. From this RNA, 110 Affymetrix gene chips were done on the same platform (HG-U133A plus) than the Immunome using the HG-U133A GeneChip 3′ IVT Express Kit. The raw data was normalized using the GCRMA algorithm. Finally, the log2 intensities of the gene expression data were used for further analysis.

Patients were sorted based on the CD8A expression level and high (Hi) and low (Lo) expressed patient group were defined (optimal cut-off). In the same way Hi and Lo patient groups were defined based on TIM3 (HAVR2) expression. Survival of patient with high expression of both genes CD8A and TIM3 (HiHi) was compared to the rest of the cohort (Kaplan-Meier (KM) curves for Disease-Free-Survival). A logrank p-value smaller than 0.05 was considered significant.

2) In addition, the repository Gene Expression Omnibus (Subramanian A et al., 2005) was screened for publicly available cancer data. Colon cancer gene expression data matrix (Affymetrix Human Genome U133 Plus 2.0 Array) and clinical information from the dataset GSE17536 (Smith JJ et al 2010; Freeman TJ et al., 2012) were downloaded. Patients (n=177) were sorted based on the CD8A expression level and high (Hi) and low (Lo) expressed patient group were defined (optimal cut-off). In the same way Hi and Lo patient groups were defined based on TIM3 (HAVR2) expression. Survival of patient with high expression of both genes CD8A and TIM3 (HiHi) was compared to the rest of the cohort (Kaplan-Meier (KM) curves for Disease-Free-Survival). A logrank p-value smaller than 0.05 was considered significant.

In the same way were analysed gene expression matrices from breast cancer tumors stored in the datasets: GSE24450 (familial breast cancer, n=183, Heikkinen T et al. 2011; Muranen TA et al. 2011) and GSE21653 (invasive early breast adenocarcinoma, n=266, Sabatier R et al. 2011; Sabatier R et al. 2011). Smith JJ et al. Experimentally derived metastasis gene expression profile predicts recurrence and death in patients with colon cancer. Gastroenterology 2010 March; 138(3):958-68. PMID: 19914252. Freeman TJ et al. Smad4-mediated signaling inhibits intestinal neoplasia by inhibiting expression of β-catenin. Gastroenterology 2012 March; 142(3):562-571.e2. PMID: 22115830. Heikkinen T et al. Variants on the promoter region of PTEN affect breast cancer progression and patient survival. Breast Cancer Res 2011; 13(6):R130. PMID: 22171747. Muranen TA et al. Breast tumors from CHEK2 1100de1C-mutation carriers: genomic landscape and clinical implications. Breast Cancer Res 2011 Sep. 20; 13(5):R90. PMID: 21542898. Sabatier R et al. A gene expression signature identifies two prognostic subgroups of basal breast cancer. Breast Cancer Res Treat 2011 April; 126(2):407-20. PMID: 20490655. Sabatier R et al. Down-regulation of ECRG4, a candidate tumor suppressor gene, in human breast cancer. PLoS One 2011; 6(11):e27656. PMID: 22110708.

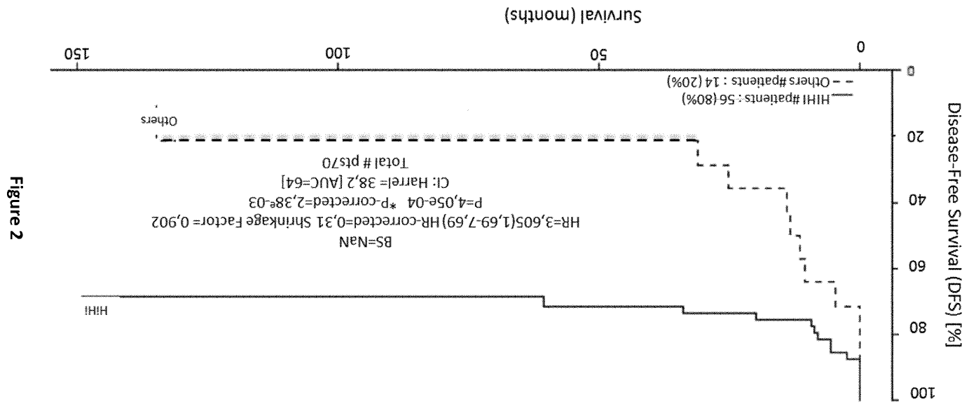

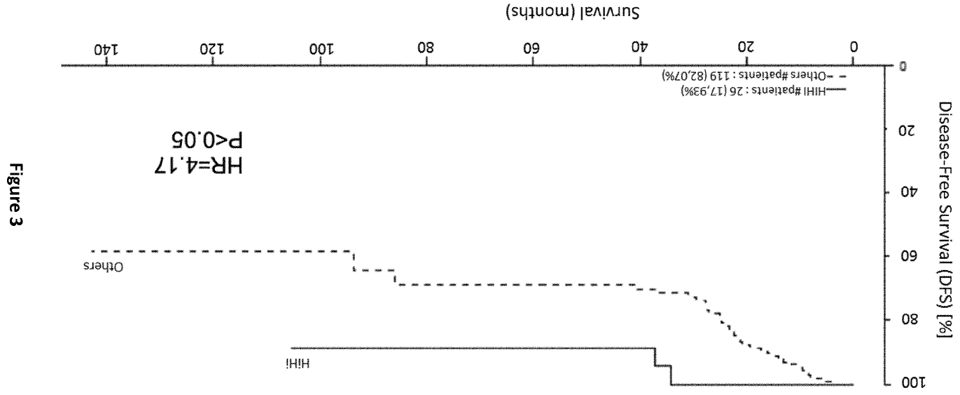

Patients with colon cancer stages I-IV were categorized into 2 groups based on based on the CD8A and TIM-3 (HAVR2) expression level. Cytotoxic T-cells gene (CD8A) and TIM-3 gene (HAVCR2) expression were measured, and categorized in high expression or low expression using the optimal-P-value cutoff. Kaplan-Meier curves were determined (see FIGS. 2-3). As shown in FIGS. 2 and 3, patients suffering from colon cancer and having both high expression level of CD8A and of HAVCR2 had better prognosis (P<0.05).

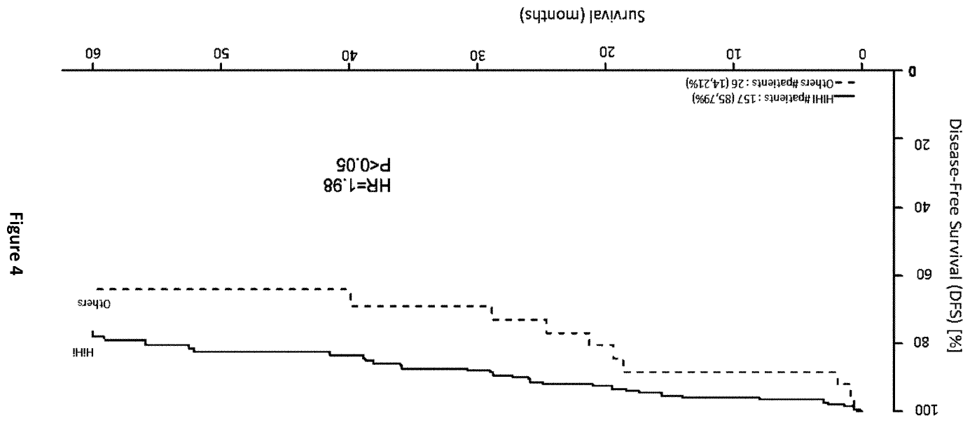

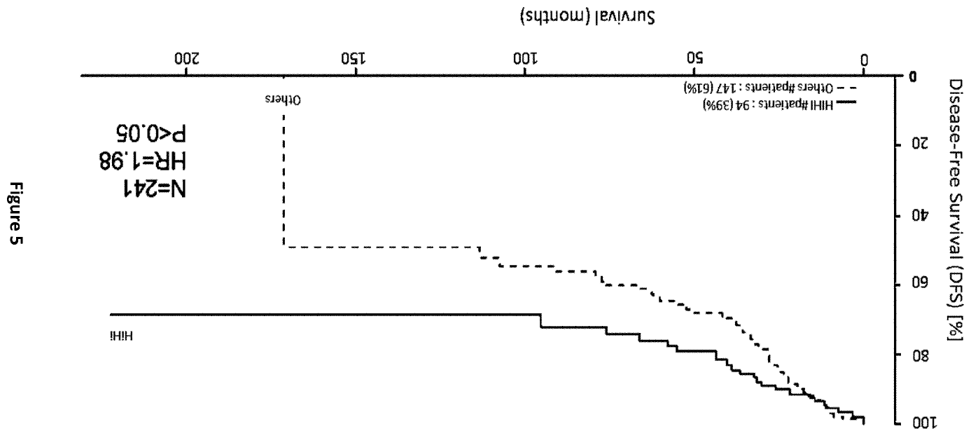

Patients with breast cancer (familial breast cancer or early stage invasive) were categorized into 2 groups based on based on the CD8A and TIM-3 (HAVR2) expression level. Cytotoxic T-cells gene (CD8A) and TIM-3 gene (HAVCR2) expression were measured, and categorized in high expression (Hi) or low expression (Lo) using the optimal-P-value cutoff Kaplan-Meier curves of disease-free survival were determined (see FIGS. 4-5). As shown in FIGS. 4 and 5, patients suffering from breast cancer and having both high expression level of CD8A and HAVCR2 had better prognosis (P<0.05).

### Example 3: The Expression Level of TIM-3 alone or in Combination with the Expression Level of a Biomarker of the Adaptive Immune Response is Predictive of a Patient's Response to Anticancer Treatment

The repository Gene Expression Omnibus (Subramanian A et al., 2005) was screened for publicly available cancer data. Colon cancer gene expression data matrix (Affymetrix Human Genome U133 Plus 2.0 Array) and clinical information from the datasets GSE40967 (Marisa L et al. 2013, n=589) and GSE31595 (n=37) were downloaded. Patients were sorted based on the CD8A expression level and high (Hi) and low (Lo) expressed patient group were defined (optimal cut-off). In the same way Hi and Lo patient groups were defined based on TIM3 (HAVR2) expression. Survival of patient with high expression of both genes CD8A and TIM3 that received adjuvant chemotherapy (YESHiHi) was compared to not treated patients with high expression of these genes (NOHiHi) and the rest of the cohort (Kaplan-Meier (KM) curves for Disease-Free-Survival). A logrank p-value smaller than 0.05 was considered significant.

Marisa L et al. Gene expression classification of colon cancer into molecular subtypes: characterization, validation, and prognostic value. PLoS Med 2013; 10(5):e1001453. PMID: 23700391.

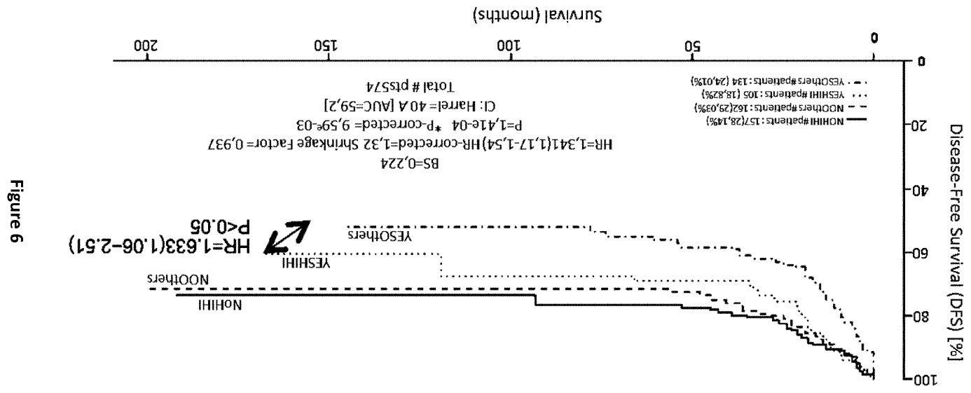

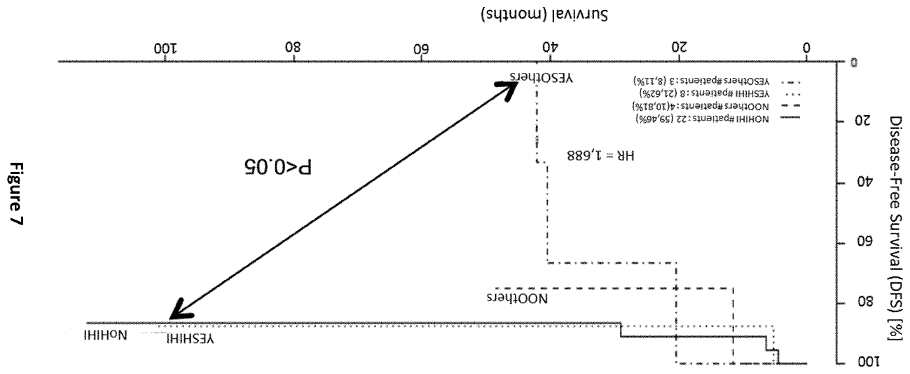

Colon cancer patients were categorized into 4 groups: patients treated with adjuvant chemotherapy or not treated with adjuvant chemotherapy, and patients categorized based on the CD8A and TIM-3 (HAVR2) expression level. Cytotoxic T-cells gene (CD8A) and TIM-3 gene (HAVCR2) expression were measured, and categorized in high expression (Hi) or low expression (Lo) using the optimal-P-value cutoff. Kaplan-Meier curves of disease-free survival were represented (see FIGS. 6 and 7). As shown in FIGS. 6 and 7, patients having both high expression level of CD8A and HAVCR2 had a better response to adjuvant chemotherapy (P<0.05).

Flow cytometry experiments were performed on fresh tumor samples (n=30 patients). Tumor infiltrating lymphocytes (TILs), normal adjacent colon infiltrating lymphocytes (NILs) and peripheral blood mononuclear cells (PBMC) were investigated (data not shown). A higher percentage of TILs co-express TIM3 and PD1 compared to NILs and PBMC (data not shown). The co-expression was observed both compartments, T helper and cytotoxic T cells. 20% of the T helper CD4+ cells co-expressed TIM3 and PD1 compared to 3.8% of the NILs and 0.1% of the PBMC. The percentage of co-expression was even higher in the cytotoxic CD8+ T cell compartment where more than 30% of TILs co-expressed TIM3 and PD1. Only 0.4% and 2% of the cytotoxic PBMC and NILs co-expressed TIM3 and PD1, respectively.

1. A method for treating a patient suffering from a solid cancer, said method comprising the steps of evaluating the prognosis of said patient by of determining, in a tumour tissue sample obtained from said patient, the ratio of the expression level of TIM-3 to the expression level of a biomarker of the adaptive immune response; and treating the patient in view of said prognosis.

2. The method according to claim 1, said method comprising the following steps:
i) determining the expression level of TIM-3 and that of a biomarker of the adaptive immune response in the centre of the tumour (CT) in a tumour tissue sample obtained from said patient;
ii) determining the ratio of the expression level of TIM-3 to the expression level of said bionaarker in the centre of the tumour;
iii) comparing said ratio with a predetermined reference value;
iv) providing a good prognosis when said ratio is higher than the predetermined reference value and treating the patient accordingly.

3. The method according to claim 1, said method comprising the following steps:
i) determining the expression level of TIM-3 and that of a biomarker of the adaptive immune response in the invasive margin (IM) in a tumour tissue sample obtained from said patient;
ii) determining the ratio of the expression level of TIM-3 to the expression level of said biomarker in the invasive margin of the tumour;
iii) comparing said ratio with a predetermined reference value;
iv) providing a bad prognosis when said ratio is higher than the predetermined reference value and treating the patient accordingly.

4. A method for determining the prognosis of a patient suffering from solid cancer comprising the steps of;
i) measuring, in a tumour tissue ample obtained from said patient, the expression level of TIM-3;
ii) comparing said TIM-3 expression level with a predetermined reference value;
iii) providing a good prognosis when expression level of TIM-3 is higher than the predetermined reference value and treating the patient accordingly.

5. The method according to claim 4, wherein in step i) the expression level of a biomarker of the adaptive immune response is further measured, in step ii) said TIM-3 expression level and said biomarker expression level are compared with predetermined reference values, and wherein a good prognosis is provided when both the expression level of TIM-3 and of the hiomarker are higher that their predetermined reference values.

6. A method for treating a patient suffering from solid cancer said method comprising the steps of:
i) measuring, in a tumour tissue sample obtained from said patient, the expression level of TIM-3 and that of a biomarker of the adaptive immune response;
ii) comparing said TIM-3 expression level and said biomarker expression level with predetermined reference values;
iii) assessing that the patient will advantageously receive an anticancer treatment when both the expression level of TIM-3 and of said biomarker are higher that their predetermined reference values; and iv) treating the patient according to the results obtained in step iii).

7. The method according to claim 6, wherein said anticancer treatment is an immunotherapy agent.
